(19) United States
(12) Patent Application Publication
(10) Pub. No.: US 2025/0029688 A1
(43) Pub. Date: Jan. 23, 2025

(54) SYSTEMS AND METHODS FOR CLINICAL EVENT EVALUATION

(71) Applicant: EDWARDS LIFESCIENCES CORPORATION, Irvine, CA (US)

(72) Inventors: Saman Parvaneh, Danver, MA (US); Yanjun Chen, Santa Ana, CA (US); Ihsan J. Bermani (formerly Hasan), Lake Forest, CA (US); Dawei Qian, Irvine, CA (US); Carlie Yvonne Gaunty Crowder, Thompson’s Station, TN (US); Terri Kang Johnson, San Diego, CA (US); Zaniar Ardalan, Tucson, AZ (US); Elena Christy Khury, Temecula, CA (US)

(21) Appl. No.: 18/904,572

(22) Filed: Oct. 2, 2024

Related U.S. Application Data
(63) Continuation of application No. PCT/US2023/017303, filed on Apr. 3, 2023.
(60) Provisional application No. 63/374,395, filed on Sep. 2, 2022.
(60) Provisional application No. 63/327,299, filed on Apr. 4, 2022.

(51) Int. Cl. G16H 10/20; G16H 50/50

(52) U.S. Cl. CPC G16H 10/20; G16H 50/50

(57) ABSTRACT

Systems and methods for assessment of clinical events within clinical trials are provided. Computational models can be utilized to evaluate clinical events. Features to be utilized within a computational model can be derived from clinical reports and clinical data.

### CROSS REFERENCE TO RELATED APPLICATIONS

The current application claims the benefit of and priority to PCT Application No. PCT/US2023/017303, entitled “SYSTEMS AND METHODS FOR CLINICAL EVENT EVALUATION,” filed Apr. 3, 2023, which claims the benefit of and priority to U.S. Provisional Patent Application No. 63/374,395 entitled “SYSTEMS AND METHODS FOR CLINICAL EVENT EVALUATION” filed Sep. 2, 2022 and to U.S. Provisional Patent Application No. 63/327,299 entitled “AUTOMATIC ENDPOINT ADJUDICATION USING ARTIFICIAL INTELLIGENCE” filed Apr. 4, 2022. The disclosures of PCT Application No. PCT/US2023/017303, U.S. Provisional Patent Application No. 63/374,395, and U.S. Provisional Patent Application No. 63/327,299 are hereby incorporated by reference in their entirety for all purposes.

### TECHNOLOGICAL FIELD

The disclosure is generally directed to systems and methods for evaluation of clinical events within clinical trials.

### BACKGROUND

Clinical trials are medical research assessments that evaluate medical, surgical, or behavioral intervention in human individuals and are typically overseen by a governmental regulatory agency. Newly developed treatments are assessed in these controlled studies. Accordingly, clinical trials assess new clinical procedures (e.g., surgery), a medical device utilized during a treatment (e.g., surgical tool or an administration device), a prosthetic device, or a medicinal product. The goal of a clinical trial is to determine whether a new treatment is more effective and/or less harmful than the current standard of care.

Clinical events are medically related events that a patient experiences within a clinical trial, often having adverse or unexpected consequence on the patient. Clinical events require further evaluation to determine whether the clinical event is related to the clinical trial treatment. Often, to ensure unbiased reporting, clinical events are adjudicated by a clinical event committee composed of medical experts that are unaffiliated with the clinical trial including sponsor.

### SUMMARY

Systems and methods for evaluating a clinical event can comprise utilization of at least one clinical form and/or clinical data. Features can be generated from the at least one clinical form and/or clinical data. The generated features can be utilized in a computational model to evaluate a clinical event to determine whether the clinical event is related to a clinical trial treatment.

In an aspect, a computational method is utilized to evaluate a clinical event. The method comprises generating features from at least one report associated with a patient within a clinical trial that has undergone a clinical event. The method further comprises entering the features into a predictive computational model to yield an evaluation of the clinical event.

In some implementations, the method further comprises receiving the at least one report associated with a patient within a clinical trial that has undergone a clinical event.

In some implementations, the clinical event is one of: an adverse event, a serious adverse event, an adverse reaction, or a suspected unexpected serious adverse event.

In some implementations, the clinical event comprises: hospitalization or rehospitalization, disability, congenital anomaly, required intervention, allergic reaction, blood dyscrasias, seizures or convulsions, development of drug dependence or drug abuse, death, or a cardiovascular related event.

In some implementations, the cardiovascular related event is one of: transient ischemic attack (TIA), bleeding, myocardial infarction, arrythmia or conduction disturbances, structural valve deterioration, endocarditis, prosthetic valve dysfunction, thrombosis, and coronary obstruction.

In some implementations, the clinical trial is assessing: a clinical procedure performed, a medical device utilized during a treatment, a prosthetic device, a system for performing clinical procedure, or a medicinal product.

In some implementations, the at least one received report is a case report form. The features generated from the case report form comprises a categorical feature derived from a data entry.

In some implementations, the at least one received report is a narrative or a source document. The features generated from the narrative or the source document comprises a categorical feature or a numerical value feature yielded from natural language processing.

In some implementations, data is also received along the at least one report, wherein the generated features comprise: a demographic feature, a risk score feature, a baseline measurement, an echocardiography feature, a text feature, or a time period between a clinical trial procedure and clinical event feature.

In some implementations, the generated features comprise: a Medical Dictionary for Regulatory Activity (MedDRA) preferred term, a Medical Dictionary for Regulatory Activity system organ class, or a time period between a clinical trial procedure and clinical event.

In some implementations, the generated features comprise: a Medical Dictionary for Regulatory Activity preferred term, a Medical Dictionary for Regulatory Activity system organ class, and a time period between a clinical trial procedure and clinical event.

In some implementations, the predictive computational model is a regression-based model or a classification-based model.

In some implementations, the evaluation of the clinical event is yielded in lieu of an adjudication by a clinical event committee.

In some implementations, the evaluation of the clinical event is yielded to assess an adjudication by a clinical event committee.

In some implementations, the features for entering into the predictive computational model are selected based on a correlation with evaluating the clinical event.

In some implementations, the features for entering into the predictive computational model are selected based on a lack of collinearity with other features.

In some implementations, the features for entering into the predictive computational model are selected based on an ability to predict.

In some implementations, the features for entering into the predictive computational model are selected based on importance as determined by the Shapley additive explanation method.

In some implementations, the computational method is for evaluating whether a rehospitalization is a cardiovascular related event or a non-cardiovascular event. The computational method comprises receiving at least one report associated with a patient within a clinical trial that has undergone a rehospitalization. The clinical trial is assessing a cardiac prosthetic or a procedure associated with the cardiac prosthetic. The method comprises generating features from the at least one received report. The method comprises entering the features into a predictive computational model to yield whether the rehospitalization was cardiovascular related or non-cardiovascular related.

In some implementations, the computational method is performed on a computational processing system. The system comprises a processor system and a memory system. The memory system comprises one or more applications that direct the processor to perform the computational method.

In some implementations, a computer-implemented method is performed. The computer-implemented method comprises storing a plurality of features of observed clinical endpoints and adjudication classifications of the observed clinical endpoints. The computer-implemented method further comprises training a machine language model to predict adjudication classification of an input observed clinical endpoint based on the plurality of features.

In some implementations, the computer-implemented method further comprises predicting adjudication classification of the input observed clinical endpoint. The predicting comprises applying observed features of the input observed clinical endpoint to the machine language model.

In some implementations, a computer-implemented method comprises storing a plurality of features of an input observed clinical endpoint. The computer-implemented method further comprises predicting an adjudication classification of the input observed clinical endpoint based on the plurality of features. The predicting comprises applying the features to a machine language model trained to predict adjudication classification of the input observed clinical endpoint based on adjudication classifications of past observed clinical endpoints.

In some implementations, a computer-implemented method comprises storing feature values of a plurality of features of an input observed clinical endpoint. The computer-implemented further comprises predicting an adjudication classification of the input observed clinical endpoint based on the feature values of the plurality of features of the input observed clinical endpoint. The predicting comprises applying the feature values of the plurality of features of the input observed clinical endpoint to a machine language model trained to predict adjudication classification of the input observed clinical endpoint based on adjudication classifications and feature values of the plurality of features of past observed clinical endpoints.

In some implementations, a computer-implemented method further comprises extracting values of at least one of the features via natural language processing.

In some implementations, the features comprise days between procedure and event of observed clinical endpoint.

In some implementations, a computer-implemented method further comprises creating training and test data sets via applying stratified splitting.

In some implementations, the method further comprises applying an advanced boosting algorithm.

In some implementations, the features comprise demographics features, risk scores features, baseline measures, echocardiography features, and/or text features.

In some implementations, the features comprise race, age, sex, and BMI.

In some implementations, the features comprise heartrate, rhythm, LVEDD, LVEF, AOVMG, and/or NYHA.

In some implementations, the features comprise AE term, AE MEDDRA preferred term (PT), and/or AE description.

In some implementations, the features comprise a MedDRA preferred term.

In some implementations, the features comprise a MedDRA System Organ Classes.

In some implementations, the computer-implemented method further comprises choosing a feature based on collinearity.

In some implementations, the input observed clinical endpoint comprises rehospitalization.

In some implementations, the adjudication classification comprises whether the input observed clinical endpoint is cardiovascular related.

In some implementations, the adjudication classification comprises whether the input observed clinical endpoint is heart failure related.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1A provides an example of a computational method for evaluating a clinical event.

FIG. 1B provides potential uses of computational method for evaluating a computational event.

FIG. 2 provides a conceptual illustration of a computational processing system for evaluating a clinical event.

FIG. 3 provides details of an experimental clinical trial in which rehospitalization was adjudicated to determine whether the rehospitalization was cardiovascular (CV) related or non-cardiovascular (NCV) related.

FIG. 4 provides a data graph showing the top twenty covariates assessed for evaluating cardiovascular related rehospitalization.

FIG. 5 provides an example of variables that can comprise data features to be utilized within a computational model to evaluate cardiovascular related rehospitalization.

FIGS. 6A and 6B provide an example of performance results of a computational model that evaluates cardiovascular related rehospitalization.

FIG. 7 provides an example of a small set of variables that can comprise data features to be utilized within a streamlined computational model to evaluate cardiovascular related rehospitalization.

FIG. 8 provides a schematic and data for evaluating a streamlined computational model that evaluates cardiovascular related rehospitalization.

FIGS. 9A and 9B provide performance results of a streamlined computational model that evaluates cardiovascular related rehospitalization.

FIG. 10 provides results of feature importance utilized within a streamlined computational model that evaluates cardiovascular related rehospitalization.

FIG. 11 provides the results of using a computational model as quality control on evaluations provided by a clinical event committee.

FIG. 12 provides a table of computational models that have been trained to evaluate various clinical event types.

### DETAILED DESCRIPTION

The current disclosure details systems and methods to evaluate clinical events of patients within clinical trials utilizing data that is associated with the clinical event. Clinical event data features can be derived and utilized to evaluate the clinical event to determine one or more causes of the event. Accordingly, systems and methods can evaluate the circumstances surrounding a clinical event to determine whether the clinical event was related to a treatment being assessed within a clinical trial. In various implementations, the clinical event is assessed to determine if it is related to a clinical procedure performed, a medical device utilized during a treatment, a system for performing clinical procedure, a prosthetic device, or a medicinal product.

Novel systems and methods provide for evaluating clinical events utilizing data associated with the clinical event. Accordingly, a clinical event can be evaluated without the adjudication of a clinical event committee, which can enhance evaluation consistency, improve turnaround time, and reduce costs. In some situations, the various systems and methods of this disclosure can be utilized to assess the quality of an adjudication outcome provided by a clinical event committee.

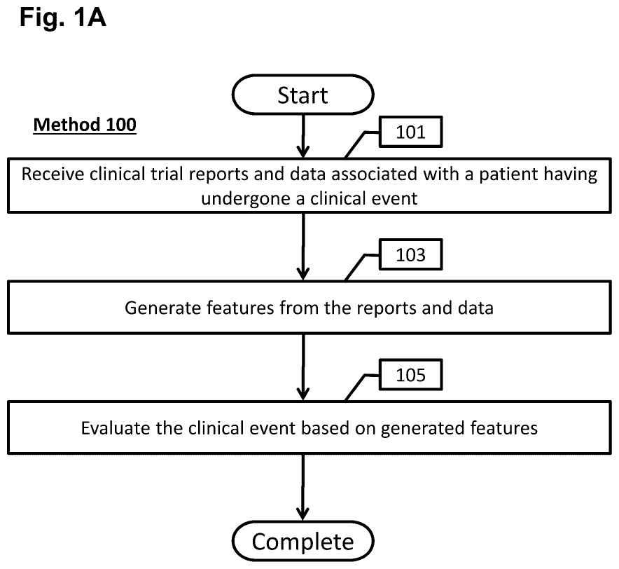

A method for evaluating a clinical event is provided in FIG. 1, which can be implemented as a computational process. Method 100 receives (101) reports and data associated with a patient having undergone a clinical event. A clinical event is an event experienced by a patient within a clinical trial and is defined by the clinical trial protocol. Clinical events include (but are not limited to) an adverse event (AE), a serious adverse event (SAE), an adverse reaction (AR), and a suspected unexpected serious adverse event (SUSAR). The precise definition of various clinical events will vary between regulatory agencies of different jurisdictions, as is determinable via the regulatory agency the clinical trial is registered within. Examples of clinical events include (but are not limited to) hospitalization or rehospitalization, disability, congenital anomaly (as related to pregnancy), required intervention (as to prevent further impairment or damage), allergic reaction (e.g., allergic bronchospasm), blood dyscrasias, seizures or convulsions, development of drug dependence or drug abuse, death, and cardiovascular related events. Cardiovascular related events include (but are not limited to) transient ischemic attack (TIA), stroke, bleeding, myocardial infarction, arrythmia or conduction disturbances, structural valve deterioration, endocarditis, prosthetic valve performance, prosthetic valve dysfunction, prosthetic valve reintervention, thrombosis, vascular access, and coronary obstruction.

Reports and data to be received include any patient clinical notes or any patient clinical data obtained in relation to the clinical trial and/or clinical event. Examples of reports include (but are not limited to) a case report form (CRF), a narrative (summary describing the event(s) over the course of the trial and/or patient medical history), and a source document (original records of clinical findings or observations). Data can include any clinical data obtained from the patient. Common data collected include (but is not limited to) cardiovascular data, cognitive data, blood panel data, urine panel data, biopsy data, and medical imaging.

Method 100 also generates (103) features from the reports and data, in which the generated features are utilized within a predictive computational model to evaluate (105) the clinical event. Features can be generated in various methods, as dependent on the contents of the reports and data collected. Any feature found to be correlative with or provide a predictive ability to evaluate the clinical event can be utilized. Furthermore, features can be weighted as appropriate to provide predictive ability. In some implementations, one-hot encoding is utilized, which may be beneficial when a lot of features are utilized within the predictive model.

To evaluate a clinical event, various predictive computational models can be utilized, including (but not limited to) regression-based or classification-based models. Generally, regression-based models provide a score that indicates a likelihood that a clinical trial is related to the clinical event that occurred and classification-based models provide discrete result (e.g., clinical event IS or IS NOT related to a clinical trial). Regression-based models include (but are not limited to) LASSO regression, ridge regression, k-nearest neighbors, elastic net, least angle regression (LAR), and random forest regression. Classification-based models include (but are not limited to) support vector machines (SVMs), decision trees, random forests, and naïve Bayes. In some implementations, a regression-based model or a classification-based model is regularized. In some implementations, a regression-based model or a classification-based model is gradient boosted.

Various numerical data points can be utilized as numerical features or transformed and then utilized as features. For instance, some numerical data can be transformed into another numerical scale. In some instances, numerical data can be transformed into a categorical status and the categorical status can be utilized as feature within the predictive computational model.

Features can also be generated from various forms documents. Data entries within a CRF can be utilized as a categorical feature. For instance, an associated term or a preferred term can be utilized as feature. Preferred terms can be chosen by a coder based on its representation of an event. In some instances, data entries within a CRF can be transformed into a numerical feature.

Utilizing natural language processing, narratives and source documents can be processed and analyzed to yield key elements that signify the report and the categorized appropriately. The categorical status can then be utilized as feature within the predictive computational model. Alternatively, natural language processing can be used to extract a numerical value from narratives and source documents, which can then be utilized as a feature within the predictive model.

Features that can be utilized include (but are not limited to) demographic features, risk score features, baseline measurements, echocardiography features, text features, and a time period between clinical trial procedure and clinical event. Demographic features can include (but are not limited to) race, age, sex, BMI. Echocardiography and physiological features include (but are not limited to) heart rate, heart rhythm, left ventricular end-diastolic diameter (LVEDD), left ventricular ejection fraction (LVEF), aortic valve mean gradient (AVMG or AOVMG), and NYHA heart failure classification. Text features include (but are not limited to) AE term, AE description, Medical Dictionary for Regulatory Activity (MedDRA) preferred term, MedDRA system organ class.

To assess a computational model, the ability of the computational model to accurately evaluate a clinical event from the selected features can be determined. Accordingly, various computational model architectures can be assessed and the architectures with better detection performance (e.g., accuracy) of an evaluation can be selected. In addition, various combinations of one or more features can be assessed and the one or more features with better prediction of an evaluation can be selected.

Computational models for clinical event evaluation can be implemented in various ways, which can be dependent on CEC availability (FIG. 1B). For instance, when CEC adjudication is available, predictive computational models can be further developed and/or fine-tuned. In addition, when CEC adjudication is available, predictive computational models can be used as a quality control can be performed on CEC adjudication. When CEC adjudication is unavailable, a predictive computational model can evaluate a clinical event without human input (fully automated evaluation). Or, in some instances, a predictive computational model can evaluate a clinical event and based on the outcome, human input can be provided (semi-automated evaluation).

In some implementations, a clinical event is evaluated via the computational process in lieu of an adjudication by a clinical event committee. In some implementations, a clinical event is evaluated via the computational process in combination with another determination. For instance, in some implementations, a clinical event is evaluated via the computational process and a medical professional, such as (for example) clinical trials safety officer or a clinical events committee (CEC).

In some instances, a clinical event is evaluated via the computational process and yields a determination that has low confidence (e.g., below a confidence threshold). Or, in some instances, a clinical event is evaluated via the computational process and yields a determination that the reports and data provided cannot yield a determination. In some instances, the computational process may recognize unknown patient types or a unfamiliar scenario, such as (for example) missing information, changes in data distribution, and shifting data patterns. When a computational process yields a low confidence determination or cannot yield a determination, the computational process can flag that the clinical event should be further evaluated by a medical professional, such as (for example) clinical trials safety officer or a clinical events committee (CEC). In some implementations, the model does not compute a determinate result but instead provides a result that is declared indeterminate or unsure.

In some implementations in which a CEC adjudicates a clinical event, the event is further evaluated via the computational process, which can be utilized as a confirmation or a check on the decision by a CEC. The evaluation by the computational process can determine whether the adjudication is proper, consistent, and/or accurate, providing an unbiased independent check of the committee.

When a patient's clinical event is evaluated and the relationship to a clinical trial is determined, the evaluation is utilized within a report assessing the clinical trial and more specifically assessing a clinical procedure performed, a medical device utilized during a treatment, a prosthetic device, a system for performing clinical procedure, or a medicinal product.

While specific examples of methods to evaluate a clinical event are described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods to evaluate a clinical event appropriate to the requirements of a given application can be utilized in various implementations.

### Feature Selection

As explained in the previous section, data are used as features to construct a computational model that is then used to evaluate a clinical event and its relationship to a clinical trial. Data used within the model can be selected by a number of ways. In some implementations, data features are selected by which data provide strong correlation with clinical event evaluation. In some implementations, data features are determined using a computational model, which can determine which data features provide good prediction ability. In some implementations, data features are selected based on practicality, case of obtaining the data, and/or commonality of data feature among data sources.

Data features can be identified and/or selected by several methods. In some instances, features that are relevant based on the clinical significance of the clinical trial and/or the clinical event are selected. In some instances, features are selected based on a high level of correlation or a high level of collinearity with outcome or performance to predict the clinical event evaluation. Accordingly, a strength of relationship between a feature and a clinical event evaluation can be determined. Many statistical methods are known to determine correlation strength (e.g., correlation coefficient), including linear association (Pearson correlation coefficient), Kendall rank correlation coefficient, and Spearman rank correlation coefficient. Collinearity can be determined by a correlation matrix and/or variance inflation factor (VIF). In some instances, computational models can identify features based on their cost functions. Computational models for selecting features include (but are not limited to) LASSO, elastic net, and ridge regression, which can identify features using weights or coefficients based on their performance. Importance of features can be determined by the Shapley additive explanation (SHAP) method. The computational models for identification of useful features can be different (or the same) models than the predictive models used to provide an evaluation of the clinical event. In some instances, a computational approach can search for all possible features and identify which features are most sensitive. Some computational approaches to search for features and identify sensitivity include (but are not limited to) restrictive to recursive feature elimination, and information gain criteria. In some situations, an ensemble approach is utilized that combines multiple models for feature selection and model development. In any approach, an appropriate computational model can be selected that results in a number of features that is manageable yet still provide a robust prediction. For instance, constructing predictive models from large numbers of data features may have overfitting issues. Likewise, too few features can result in less prediction power.

### Model Training

A computational model for predicting whether a clinical event is associated with a treatment being assessed within a clinical trial can be trained using prior clinical trial data. The clinical trial can be any clinical trial having a set of patients within the trail that has undergone a clinical event and had that clinical event evaluated. For each patient that underwent the clinical event, reports and data associated with the patient can be collected, especially data associated with the clinical event itself. The collected data can further include any demographic data of the patient or any data associated with the clinical trial treatment (and responses to treatment). In some implementations, the collected data includes medical data unassociated with the clinical trial but may be associated the disorder to be treated. In some implementations, any medical data of a patient is collected such that comprehensive view of the patient's health can be assessed and linked with the patient and/or the clinical event experienced by the patient. In addition to medical data, the clinical event adjudication result for each patient is collected and linked with medical data acquired. Using the medical data and clinical event adjudication result of each patient in the set, a computational model can be trained to predict whether clinical event of the patient is related to clinical trial treatment based on the medical data associated with the patient.

Medical data can be derived from medical reports, medical test results, and/or any source that provides an indication of the patient's medial history. The reports and data collected can include the reports and data associated with the clinical event itself. In some implementations, the reports and data collected include demographic data of the patient or data associated with the clinical trial treatment (and responses to treatment). In some implementations, the reports and data collected are unrelated to the clinical event and the clinical trial but are related to the patient's medical history. Examples of reports that can be collected include (but are not limited to) a case report form (CRF), a narrative (summary describing the event(s) over the course of the trial and/or patient medical history), and a source document (original records of clinical findings or observations). Examples of data that can be collected include (but is not limited to) cardiovascular data, cognitive data, blood panel data, urine panel data, biopsy data, and medical imaging.

Any computational model capable of predicting a clinical event from reports and data can be utilized, including (but not limited to) regression-based or classification-based models. Regression-based models include (but are not limited to) LASSO regression, ridge regression, k-nearest neighbors, clastic net, least angle regression (LAR), and random forest regression. Classification-based models include (but are not limited to) support vector machines (SVMs), decision trees, random forests, and naïve Bayes. In some implementations, a regression-based model or a classification-based model is regularized. In some implementations, a regression-based model or a classification-based model is gradient boosted.

Various numerical data points can be utilized as numerical features or transformed and then utilized as features. For instance, some numerical data can be transformed into another numerical scale. In some instances, numerical data can be transformed into a categorical status and the categorical status can be utilized as feature within the predictive computational model.

Features can also be generated from various forms documents. Data entries within a CRF can be utilized as a categorical feature. For instance, an associated term or a preferred term can be utilized as feature. Preferred terms can be chosen by a coder based on its representation of an event. In some instances, data entries within a CRF can be transformed into a numerical feature.

Utilizing natural language processing, narratives and source documents can be processed and analyzed to yield key elements that signify the report and the categorized appropriately. The categorical status can then be utilized as feature within the predictive computational model. Alternatively, natural language processing can be used to extract a numerical value from narratives and source documents, which can then be utilized as a feature within the predictive model.

The trained computational model can be utilized to predict whether a clinical event is associated with a clinical trial treatment. Any clinical event can be assessed, including (but not limited to) an adverse event (AE), a serious adverse event (SAE), an adverse reaction (AR), and a suspected unexpected serious adverse event (SUSAR). The precise definition of various clinical events will vary between regulatory agencies of different jurisdictions, as is determinable via the regulatory agency the clinical trial is registered within. Examples of clinical events include (but are not limited to) hospitalization or rehospitalization, disability, congenital anomaly (as related to pregnancy), required intervention (as to prevent further impairment or damage), allergic reaction (e.g., allergic bronchospasm), blood dyscrasias, seizures or convulsions, development of drug dependence or drug abuse, death, and cardiovascular related events. Cardiovascular related events include (but are not limited to) transient ischemic attack (TIA), stroke, bleeding, myocardial infarction, arrythmia or conduction disturbances, structural valve deterioration, endocarditis, prosthetic valve performance, prosthetic valve dysfunction, prosthetic valve reintervention, thrombosis, vascular access, and coronary obstruction.

### Computational Processing System

A computational processing system to evaluate a clinical event in accordance with the various methods and processes of the disclosure typically utilizes a processing system including one or more of a CPU, GPU and/or neural processing engine. As described herein, clinical reports and/or data related to a clinical event can be received. Further, the features can be extracted from the clinical reports. The computational processing system can be implemented on any appropriate computing device such as (but not limited to) a desktop computer, a remote server, a tablet and/or portable computer.

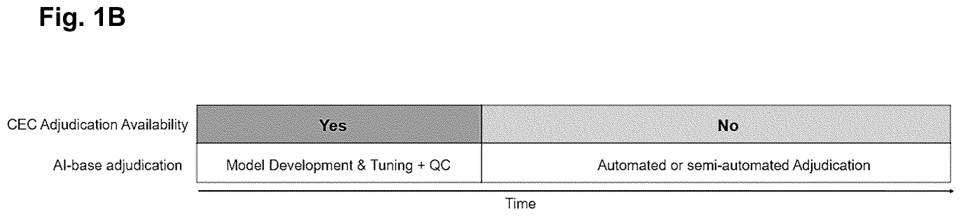

An example of a computational processing system that can be utilized to perform the various methods and processes of the disclosure is illustrated in FIG. 2, which depicts a computational system to evaluate a clinical event. Computational processing system 110 includes a processor system 112, an I/O interface 114, and a memory system 116. As can readily be appreciated, the processor system 112, I/O interface 114, and memory system 116 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash).

In the illustrated example, memory system 116 is capable of storing various data and models. It is to be understood that the listed data and models are a representative sample of what can be stored in memory and that various memory systems may store some or all of the various data and models listed. Further, any combination of data and models can be stored, and in some implementations, various data, applications, and/or models are stored temporarily.

In some implementations, the memory system 116 can store clinical reports and data 200, which can be received. Generated features 202 can be generated from reports the received clinical reports and data 200 utilizing a natural language processing model 204, which can also be stored in memory system 116. The received clinical reports and data 200 can also be transformed or used directly as features along with the generated features 202 in a clinical event evaluation model 206, which can be stored in memory system 116. Processor system 112 is configured to execute clinical event evaluation model 206 to generate a computed categorization or score 208 indicative of the relationship of the clinical event to a clinical trial. Further, the computed categorization or score 208 can be displayed on a monitor or other screen via the I/O interface 114.

While specific computational processing systems are described above with reference to FIG. 2, it should be readily appreciated that computational processes and/or other processes utilized in the provision of clinical event evaluation can be implemented on any of a variety of processing devices including combinations of processing devices. Accordingly, computational devices should be understood as not limited to specific monitoring systems, computational processing systems, and/or specific applications and models. Computational devices can be implemented using any of the combinations of systems described herein and/or modified versions of the systems described herein to perform the processes, combinations of processes, and/or modified versions of the processes described herein.

### Data Examples

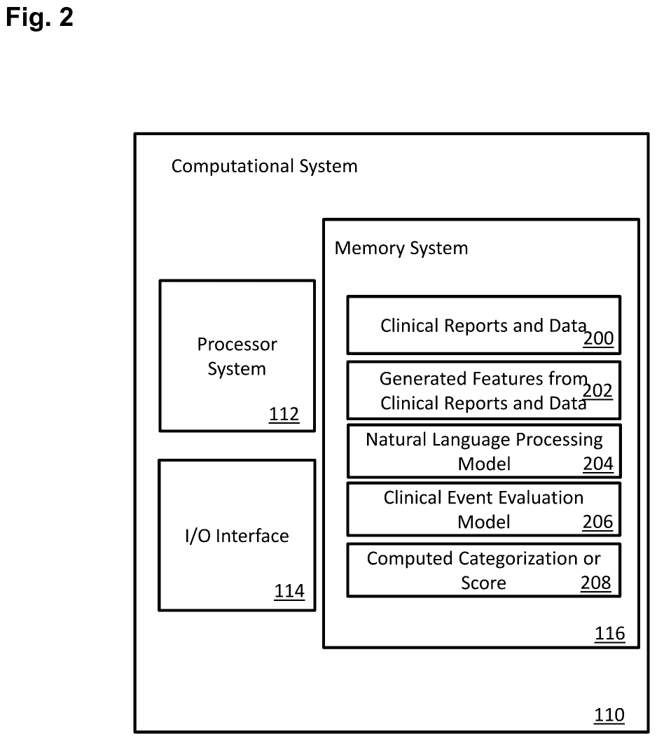

As a proof of concept, cardiovascular-related rehospitalization was utilized as a clinical event to assess the methods and processes of this disclosure. Data from the PARTNER 3 trial included over 900 AE rehospitalization cases. The cases were adjudicated by a clinical event committee, determining whether the hospitalization was cardiovascular related or non-cardiovascular related (FIG. 3). The PARTNER 3 trial was conducted to assess transcatheter aortic valve replacement with a balloon-expandable valve.

Over 200 potential variables were collected and extracted from CRFs, echocardiogram measurements, and baseline patient characteristics. The echocardiogram measurements included baseline measurements at the initiation of the clinical trial and the last measurement prior to the clinical event occurring. The variables included continuous values, ordinal values, categorical values, and narratives.

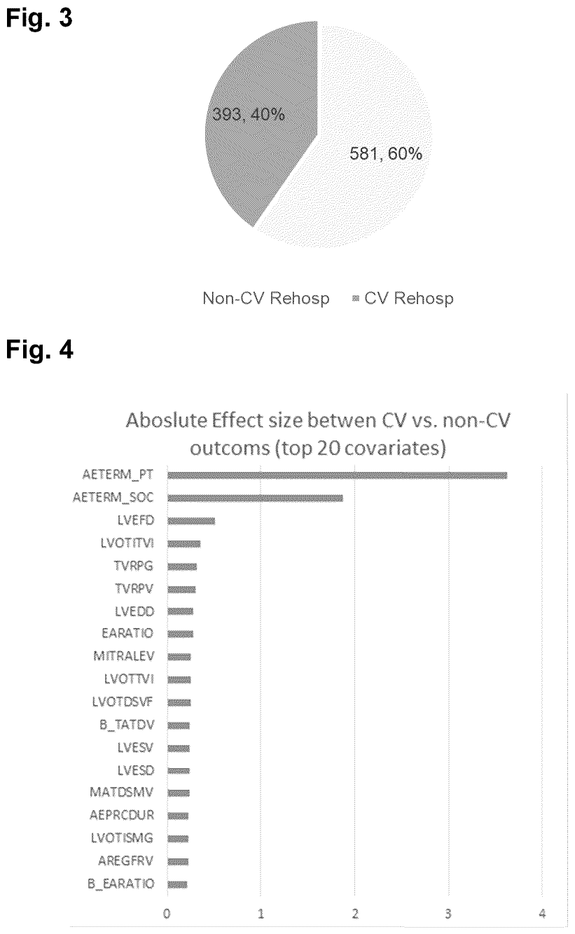

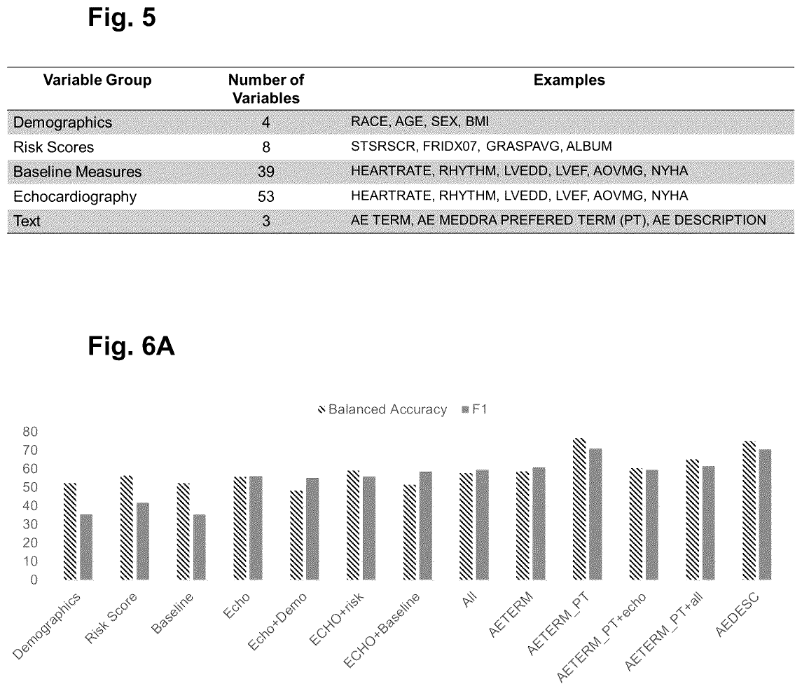

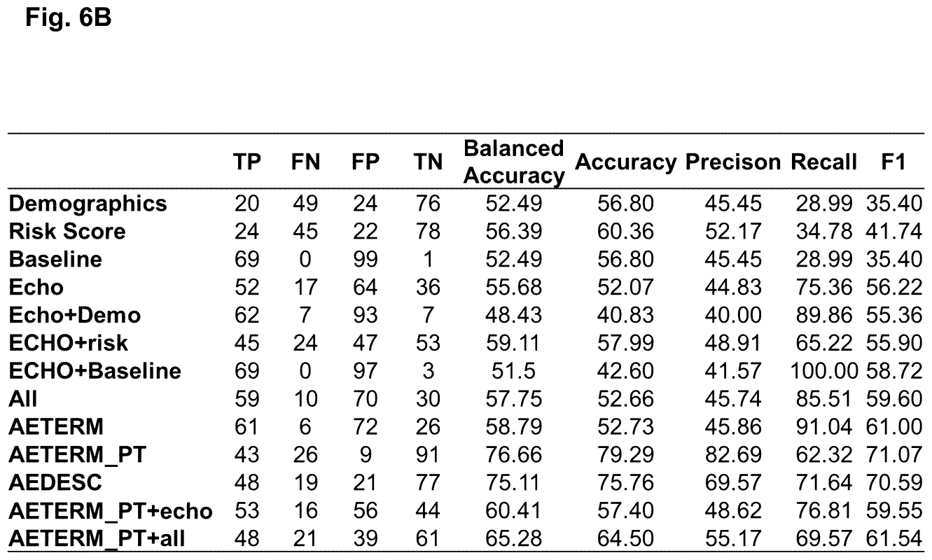

Univariable analysis was performed and the top 20 covariates are shown in FIG. 4. The top two covariates are the adverse event MedDRA preferred term (AETERM_PT) and the adverse event MedDRA system organ class (AETERM_SOC). Variables with high missing data from the patient pool and variables having high collinearity were removed, resulting in the use of just over 100 variables utilized in various combinations within various computational model (FIG. 5). The performance results of the various computational models are provided in FIGS. 6A and 6B.

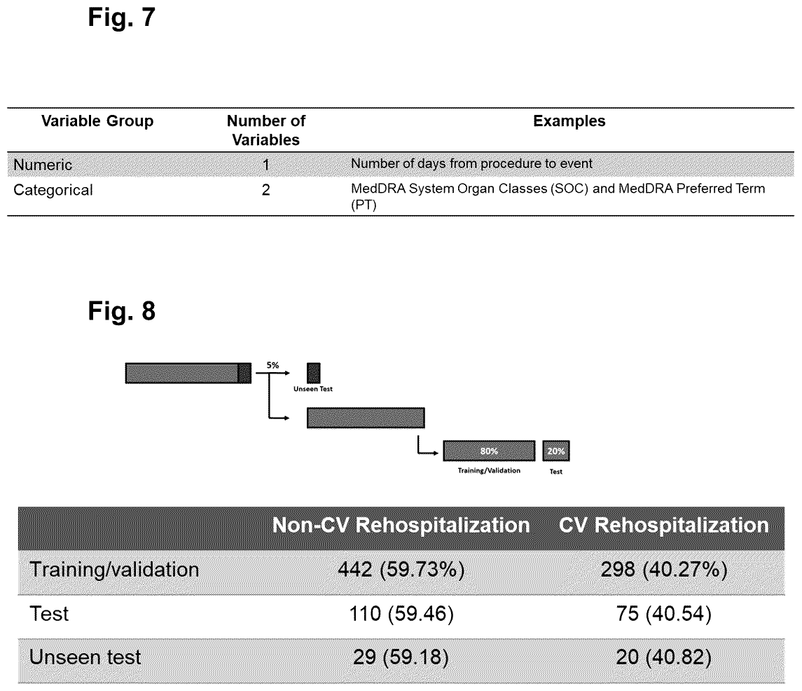

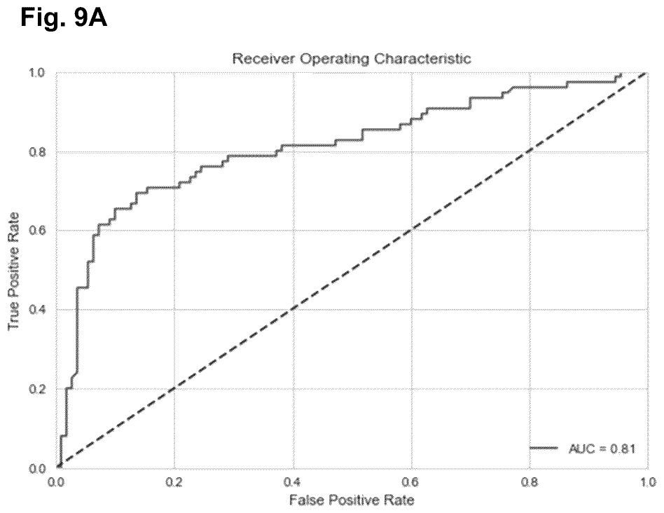

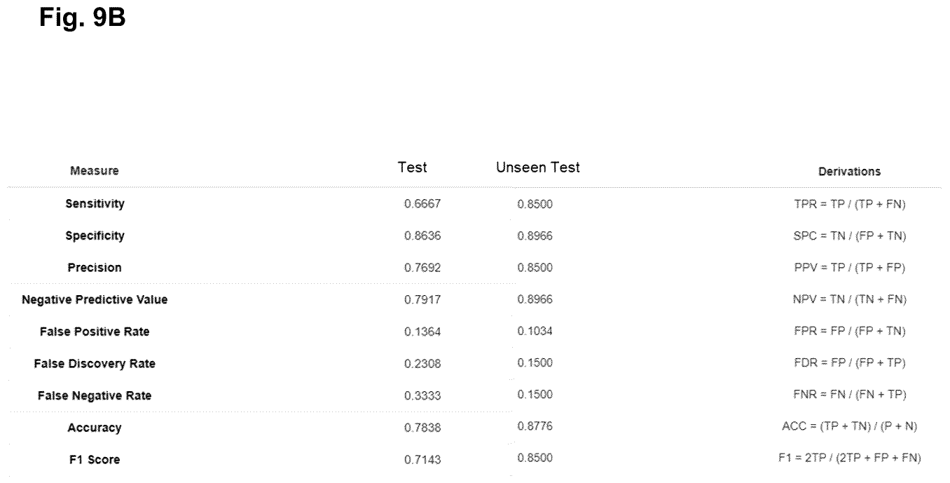

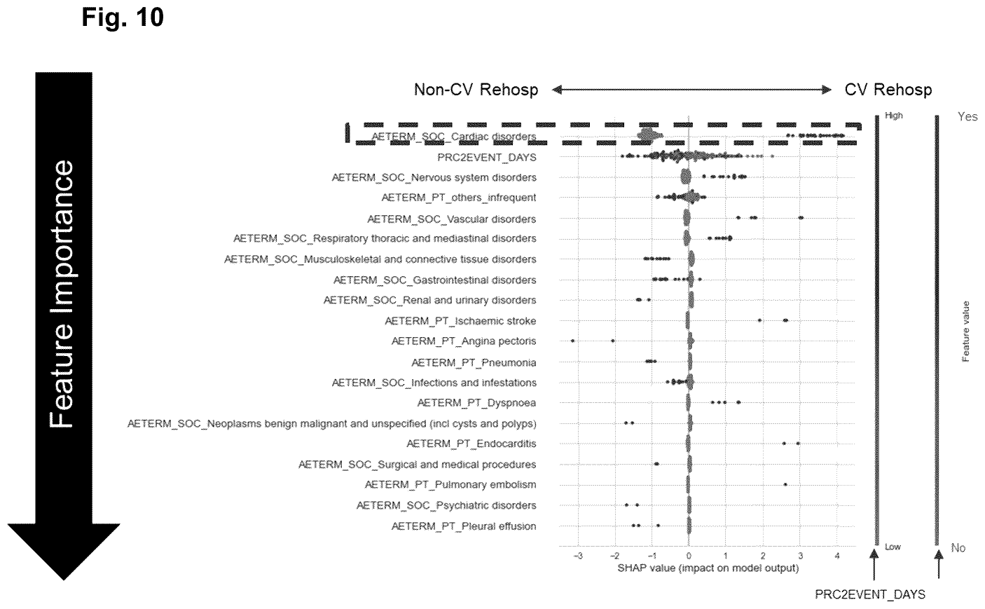

A streamlined computational model was developed utilizing three variables: days between procedure and clinical event; MedDRA preferred term, and MedDRA system organ classes (FIG. 7). In training the model, 5% of cases were withheld (unseen test) and the remaining were stratified split (80% to 20%) for training and validation data sets, respectively (FIG. 8). One-hot encoding was used for the categorical variables. Over a dozen model architectures were analyzed, and a decision tree with gradient boosting yielded the best results. The performance results of the model are provided in FIGS. 9A and 9B. The importance of the various features utilized in the model is provided in FIG. 10, which was determined utilizing the Shapley additive explanation (SHAP) method.

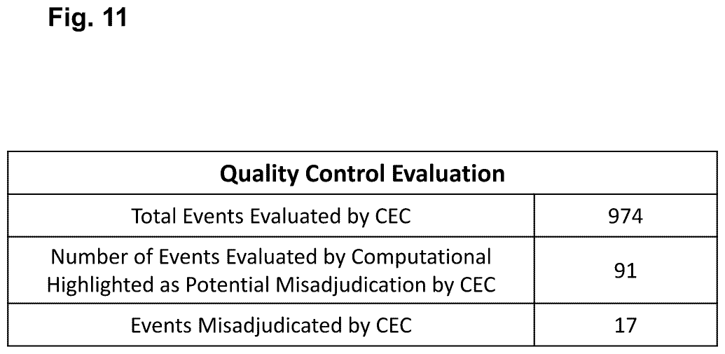

The ability of the computational process to make rehospitalization evaluations was compared with CEC adjudication (FIG. 11). A total of 974 clinical events were evaluated by a CEC and then assessed by predictive computational process. The computational process highlighted 91 of the events as potentially a misadjudication by the CEC. Upon further review, it was concluded that 17 of the 91 events were misadjudicated.

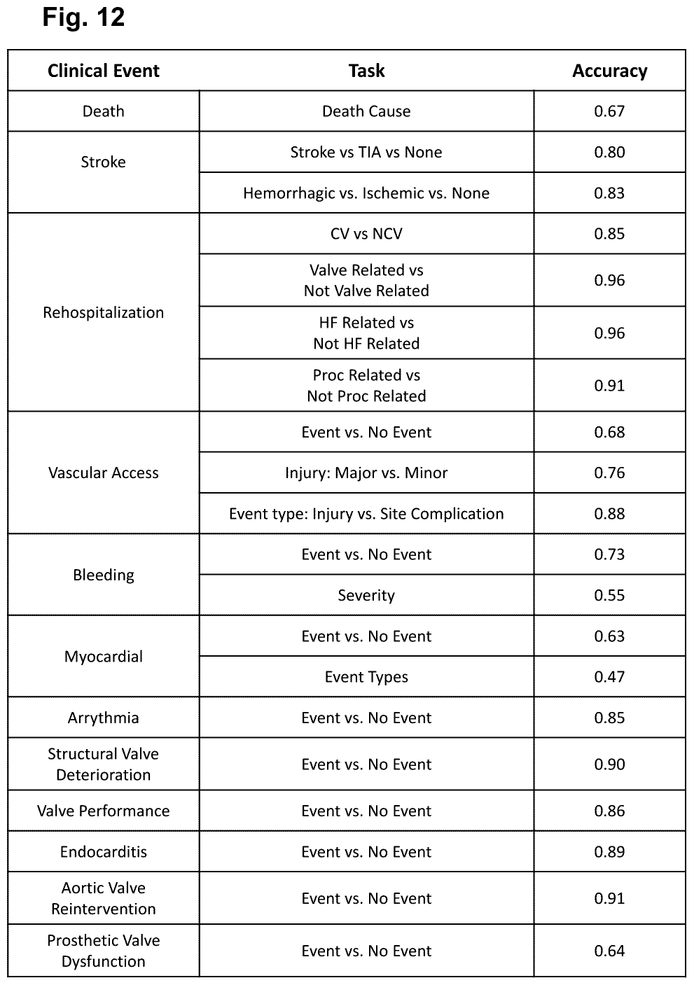

Computational models have been constructed for the following clinical events: death, stroke, rehospitalization, vascular access, bleeding, myocardial, arrythmias, structural valve deterioration, valve performance, endocarditis, aortic valve reintervention, and prosthetic valve dysfunction (FIG. 12). Each constructed model had a task associated with the clinical event was able to predict event outcome with a reasonable amount of success.

### EXAMPLES

1. An example computational method for evaluating a clinical event, comprising:

receiving, using a computational processing system, at least one report associated with a patient within a clinical trial that has undergone a clinical event;
generating, using the computational processing system, features from the at least one received report; and
entering, using the computational processing system, the features into a predictive computational model to yield an evaluation of the clinical event.

2. The example computational method of example 1, wherein the clinical event is one of: an adverse event, a serious adverse event, an adverse reaction, or a suspected unexpected serious adverse event.

3. The example computational method of example 1 or 2, wherein the clinical event comprises: hospitalization or rehospitalization, disability, congenital anomaly, required intervention, allergic reaction, blood dyscrasias, seizures or convulsions, development of drug dependence or drug abuse, death, or a cardiovascular related event.

4. The example computational method of example 3, wherein the cardiovascular related event is one of: transient ischemic attack (TIA), bleeding, myocardial infarction, arrythmia or conduction disturbances, structural valve deterioration, endocarditis, prosthetic valve dysfunction, thrombosis, and coronary obstruction.

5. The example computational method of any one of examples 1 to 4, wherein the clinical trial is assessing: a clinical procedure performed, a medical device utilized during a treatment, a prosthetic device, a system for performing clinical procedure, or a medicinal product.

6. The example computational method of any one of examples 1 to 5, wherein the at least one received report is a case report form, and wherein the features generated from the case report form comprises a categorical feature derived from a data entry.

7. The example computational method of any one of examples 1 to 6, wherein the at least one received report is a narrative or a source document, and wherein the features generated from the narrative or the source document comprises a categorical feature or a numerical value feature yielded from natural language processing.

8. The example computational method of any one of examples 1 to 7, wherein data is also received along the at least one report, wherein the generated features comprise: a demographic feature, a risk score feature, a baseline measurement, an echocardiography feature, a text feature, or a time period between a clinical trial procedure and clinical event feature.

9. The example computational method of any one of examples 1 to 8, wherein the generated features comprise: a Medical Dictionary for Regulatory Activity preferred term, a Medical Dictionary for Regulatory Activity system organ class, or a time period between a clinical trial procedure and clinical event.

10. The example computational method of any one of examples 1 to 9, wherein the generated features comprise: a Medical Dictionary for Regulatory Activity preferred term, a Medical Dictionary for Regulatory Activity system organ class, and a time period between a clinical trial procedure and clinical event.

11. The example computational method of any one of examples 1 to 10, wherein the predictive computational model is a regression-based model or a classification-based model.

12. The example computational method of any one of examples 1 to 11, wherein the evaluation of the clinical event is yielded in lieu of an adjudication by a clinical event committee.

13. The example computational method of any one of examples 1 to 12, wherein the evaluation of the clinical event is yielded to assess an adjudication by a clinical event committee.

14. The example computational method of any one of examples 1 to 13, wherein the features for entering into the predictive computational model are selected based on a correlation with evaluating the clinical event.

15. The example computational method of any one of examples 1 to 14, wherein the features for entering into the predictive computational model are selected based on a lack of collinearity with other features.

16. The example computational method of any one of examples 1 to 15, wherein the features for entering into the predictive computational model are selected based on an ability to predict.

17. The example computational method of any one of examples 1 to 16, wherein the features for entering into the predictive computational model are selected based on importance as determined by the Shapley additive explanation method.

18. An example computational method for evaluating whether a rehospitalization is a cardiovascular related event or a non-cardiovascular event, comprising:

receiving, using a computational processing system, at least one report associated with a patient within a clinical trial that has undergone a rehospitalization, wherein the clinical trial is assessing a cardiac prosthetic or a procedure associated with the cardiac prosthetic;
generating, using the computational processing system, features from the at least one received report; and
entering, using the computational processing system, the features into a predictive computational model to yield whether the rehospitalization was cardiovascular related or non-cardiovascular related.

19. The example computational method of example 18, wherein the at least one received report is a case report form, and wherein the features generated from the case report form comprises a categorical feature derived from a data entry.

20. The example computational method of example 18 or 19, wherein the at least one received report is a narrative or a source document, and wherein the features generated from the narrative or the source document comprises a categorical feature or a numerical value feature yielded from natural language processing.

21. The example computational method of example 18, 19, or 20, wherein the generated features comprise: a demographic feature, a risk score feature, a baseline measurement, an echocardiography feature, a text feature, or a time period between a clinical trial procedure and clinical event feature.

22. The example computational method of any one of examples 18 to 21, wherein the generated features comprise: a Medical Dictionary for Regulatory Activity preferred term, a Medical Dictionary for Regulatory Activity system organ class, or a time period between a clinical trial procedure and clinical event.

23. The example computational method of any one of examples 18 to 22, wherein the generated features comprise: a Medical Dictionary for Regulatory Activity preferred term, a Medical Dictionary for Regulatory Activity system organ class, and a time period between a clinical trial procedure and clinical event.

24. The example computational method of any one of examples 18 to 23, wherein the predictive computational model is a regression-based model or a classification-based model.

25. The example computational method of any one of examples 18 to 24, wherein the evaluation of the clinical event is yielded in lieu of an adjudication by a clinical event committee.

26. The example computational method of any one of examples 18 to 25, wherein the evaluation of the clinical event is yielded to assess an adjudication by a clinical event committee.

27. The example computational method of any one of examples 18 to 26, wherein features are entered into a computational model are selected based on a correlation with evaluating the clinical event.

28. The example computational method of any one of examples 18 to 27, wherein features are entered into a computational model are selected based on a lack of collinearity with other features.

29. The example computational method of any one of examples 18 to 28, wherein features are entered into a computational model are selected based on an ability to predict.

30. The example computational method of any one of examples 18 to 29, wherein features are entered into a computational model are selected based on importance as determined by the Shapley additive explanation method.

31. An example computational processing system for evaluating a clinical event, the system comprising:

a processor system; and
a memory system comprising one or more applications that can direct the processor system to:
receive at least one report associated with a patient within a clinical trial that has undergone a clinical event;
generate features from the at least one received report; and
enter the features into a predictive computational model to yield an evaluation of the clinical event.

32. The example computational processing system of example 31, wherein the clinical event is one of: an adverse event, a serious adverse event, an adverse reaction, or a suspected unexpected serious adverse event.

33. The example computational processing system of example 31 or 32, wherein the clinical event comprises: hospitalization or rehospitalization, disability, congenital anomaly, required intervention, allergic reaction, blood dyscrasias, seizures or convulsions, or development of drug dependence or drug abuse, death, or a cardiovascular related event.

34. The example computational processing system of example 33, wherein the cardiovascular related event is one of: transient ischemic attack (TIA), bleeding, myocardial infarction, arrythmia or conduction disturbances, structural valve deterioration, endocarditis, prosthetic valve dysfunction, thrombosis, and coronary obstruction.

35. The example computational processing system of any one of examples 31 to 34, wherein the clinical trial is assessing: a clinical procedure performed, a medical device utilized during a treatment, a prosthetic device, a system for performing clinical procedure, or a medicinal product.

36. The example computational processing system of any one of examples 31 to 35, wherein the at least one received report is a case report form, and wherein the features generated from the case report form comprises a categorical feature derived from a data entry.

37. The example computational processing system of any one of examples 31 to 36, wherein the at least one received report is a narrative or a source document, and wherein the features generated from the narrative or the source document comprises a categorical feature or a numerical value feature yielded from natural language processing.

38. The example computational processing system of any one of examples 31 to 37, wherein data is also received along the at least one report, wherein the generated features comprise: a demographic feature, a risk score feature, a baseline measurement, an echocardiography feature, a text feature, or a time period between a clinical trial procedure and clinical event feature.

39. The example computational processing system of any one of examples 31 to 38,wherein the generated features comprise: a Medical Dictionary for Regulatory Activity preferred term, a Medical Dictionary for Regulatory Activity system organ class, or a time period between a clinical trial procedure and clinical event.

40. The example computational processing system of any one of examples 31 to 39, wherein the generated features comprise: a Medical Dictionary for Regulatory Activity preferred term, a Medical Dictionary for Regulatory Activity system organ class, and a time period between a clinical trial procedure and clinical event.

41. The example computational processing system of any one of examples 31 to 40, wherein the predictive computational model is a regression-based model or a classification-based model.

42. The example computational processing system of any one of examples 31 to 41, wherein the evaluation of the clinical event is yielded in lieu of an adjudication by a clinical event committee.

43. The example computational processing system of any one of examples 31 to 42, wherein the evaluation of the clinical event is yielded to assess an adjudication by a clinical event committee.

44. The example computational processing system of any one of examples 31 to 43, wherein features are entered into a computational model are selected based on a correlation with evaluating the clinical event.

45. The example computational processing system of any one of examples 31 to 44, wherein features are entered into a computational model are selected based on a lack of collinearity with other features.

46. The example computational processing system of any one of examples 31 to 45, wherein features are entered into a computational model are selected based on an ability to predict.

47. The example computational processing system of any one of examples 31 to 46, wherein features are entered into a computational model are selected based on importance as determined by the Shapley additive explanation method.

48. An example computational processing system for evaluating whether a rehospitalization is a cardiovascular related event or a non-cardiovascular event, the system comprising:

a processor system; and
a memory system comprising one or more applications that can direct the processor system to: receive at least one report associated with a patient within a clinical trial that has undergone a rehospitalization, wherein the clinical trial is assessing a cardiac prosthetic or a procedure associated with the cardiac prosthetic;
generate features from the at least one received report; and
enter the features into a predictive computational model to yield whether the rehospitalization was cardiovascular related or non-cardiovascular related.

49. The example computational processing system of example 48, wherein the at least one received report is a case report form, and wherein the features generated from the case report form comprises a categorical feature derived from a data entry.

50. The example computational processing system of example 48 or 48, wherein the at least one received report is a narrative or a source document, and wherein the features generated from the narrative or the source document comprises a categorical feature or a numerical value feature yielded from natural language processing.

51. The example computational processing system of example 48, 49, or 50, wherein the generated features comprise: a demographic feature, a risk score feature, a baseline measurement, an echocardiography feature, a text feature, or a time period between a clinical trial procedure and clinical event feature.

52. The example computational processing system of any one of examples 48 to 51, wherein the generated features comprise: a Medical Dictionary for Regulatory Activity preferred term, a Medical Dictionary for Regulatory Activity system organ class, or a time period between a clinical trial procedure and clinical event.

53. The example computational processing system of any one of examples 48 to 51, wherein the generated features comprise: a Medical Dictionary for Regulatory Activity preferred term, a Medical Dictionary for Regulatory Activity system organ class, and a time period between a clinical trial procedure and clinical event.

54. The example computational processing system of any one of examples 48 to 53, wherein the predictive computational model is a regression-based model or a classification-based model.

55. The example computational processing system of any one of examples 48 to 54, wherein the evaluation of the clinical event is yielded in lieu of an adjudication by a clinical event committee.

56. The example computational processing system of any one of example 48 to 55, wherein the evaluation of the clinical event is yielded to assess an adjudication by a clinical event committee.

57. The example computational processing system of any one of examples 48 to 56, wherein features are entered into a computational model are selected based on a correlation with evaluating the clinical event.

58. The example computational processing system of any one of examples 48 to 57, wherein features are entered into a computational model are selected based on a lack of collinearity with other features.

59. The example computational processing system of any one of examples 48 to 58, wherein features are entered into a computational model are selected based on an ability to predict.

60. The example computational processing system of any one of claims 48 to 59, wherein features are entered into a computational model are selected based on importance as determined by the Shapley additive explanation method.

61. An example computer-implemented method comprising:

storing, using the computational processing system, a plurality of features of observed clinical endpoints and adjudication classifications of the observed clinical endpoints; and
training, using the computational processing system, a machine language model to predict adjudication classification of an input observed clinical endpoint based on the plurality of features.

62. The example method of example 61, further comprising:

predicting, using the computational processing system, adjudication classification of the input observed clinical endpoint, wherein the predicting comprises applying observed features of the input observed clinical endpoint to the machine language model.

63. An example computer-implemented method comprising:

storing, using a computational processing system, a plurality of features of an input observed clinical endpoint; and
predicting, using the computational processing system, an adjudication classification of the input observed clinical endpoint based on the plurality of features, wherein the predicting comprises applying the features to a machine language model trained to predict adjudication classification of the input observed clinical endpoint based on adjudication classifications of past observed clinical endpoints.

64. An example computer-implemented method comprising:

storing, using a computational processing system, feature values of a plurality of features of an input observed clinical endpoint; and
predicting, using the computational processing system, an adjudication classification of the input observed clinical endpoint based on the feature values of the plurality of features of the input observed clinical endpoint, wherein the predicting comprises applying the feature values of the plurality of features of the input observed clinical endpoint to a machine language model trained to predict adjudication classification of the input observed clinical endpoint based on adjudication classifications and feature values of the plurality of features of past observed clinical endpoints.

65. The example method of any one of examples 61 to 64, further comprising: extracting, using the computational processing system, values of at least one of the features via natural language processing.

66. The example method of any one of examples 61 to 65, wherein the features comprise days between procedure and event of observed clinical endpoint.

67. The example of any one of examples 61 to 66, further comprising: creating training and test data sets via applying stratified splitting.

68. The example method of any one of examples 61 to 67, further comprising: applying, using the computational processing system, an advanced boosting algorithm.

69. The example method of any one of examples 61 to 68, wherein the features comprise demographics features, risk scores features, baseline measures, echocardiography features, or text features.

70. The example method of any one of examples 61 to 69, wherein the features comprise demographics features, risk scores features, baseline measures, echocardiography features, and text features.

71. The example method of any one of examples 61 to 70, wherein the features comprise demographics features, risk scores features, baseline measures, echocardiography features, and/or text features.

72. The example method of any one of examples 61 to 71, wherein the features comprise race, age, sex, and BMI.

73. The example method of any one of examples 61 to 72, wherein the features comprise heartrate, rhythm, LVEDD, LVEF, AOVMG, and/or NYHA.

74. The example method of any one of examples 61 to 73, wherein the features comprise AE term, AE MEDDRA preferred term (PT), and/or AE description.

75. The example method of any one of examples 61 to 74, wherein the features comprise: MedDRA preferred term.

76. The example method of any one of examples 61 to 75, wherein the features comprise: MedDRA System Organ Classes.

77. The example method of any one of examples 61 to 76, further comprising: choosing a feature based on collinearity.

78. The example method of any one of examples 61 to 77, wherein the input observed clinical endpoint comprises rehospitalization.

79. The example method of any one of examples 61 to 78, wherein the adjudication classification comprises whether the input observed clinical endpoint is cardiovascular related.

80. The example method of any one of examples 61 to 79, wherein the adjudication classification comprises whether the input observed clinical endpoint is device related.

81. The example method of any one of examples 61 to 80, wherein the adjudication classification comprises whether the input observed clinical endpoint is procedure related.

82. The example method of any one of examples 61 to 81, wherein the adjudication classification comprises whether the input observed clinical endpoint is heart failure related.

83. An example system comprising:

one or more hardware processors; and
computer memory coupled to the one or more hardware processors, wherein the computer memory stores computer-executable instructions that, when executed by a computing system, cause the computing system to perform the method of any one of claims 59 to 80.

84. An example of one or more computer-readable media comprising computer-executable instructions that, when executed by a computing system, cause the computing system to perform the method of any one of claims 61 to 83.85. One or more computer-readable media storing a machine learning model trained according to the method of any one of claims 61 to 84.

What is claimed is:

1. A computational method for evaluating a clinical event, comprising:
receiving, using a computational processing system, at least one report associated with a patient within a clinical trial that has undergone a clinical event;
generating, using the computational processing system, features from the at least one received report; and
entering, using the computational processing system, the features into a predictive computational model to yield an evaluation of the clinical event.

2. The computational method of claim 1, wherein the clinical event is one of: an adverse event, a serious adverse event, an adverse reaction, or a suspected unexpected serious adverse event.

3. The computational method of claim 1, wherein the clinical event comprises: hospitalization or rehospitalization, disability, congenital anomaly, required intervention, allergic reaction, blood dyscrasias, seizures or convulsions, development of drug dependence or drug abuse, death, or a cardiovascular related event.

4. The computational method of claim 3, wherein the cardiovascular related event is one of: transient ischemic attack (TIA), bleeding, myocardial infarction, arrythmia or conduction disturbances, structural valve deterioration, endocarditis, prosthetic valve dysfunction, thrombosis, and coronary obstruction.

5. The computational method of any claim 1, wherein the clinical trial is assessing: a clinical procedure performed, a medical device utilized during a treatment, a prosthetic device, a system for performing clinical procedure, or a medicinal product.

6. The computational method of any claim 1, wherein the at least one received report is a case report form, and wherein the features generated from the case report form comprises a categorical feature derived from a data entry.

7. The computational method of claim 1, wherein the at least one received report is a narrative or a source document, and wherein the features generated from the narrative or the source document comprises a categorical feature or a numerical value feature yielded from natural language processing.

8. The computational method of claim 1, wherein the predictive computational model is a regression-based model or a classification-based model.

9. The computational method of claim 1, wherein the evaluation of the clinical event is yielded in lieu of an adjudication by a clinical event committee.

10. The computational method of claim 1, wherein the evaluation of the clinical event is yielded to assess an adjudication by a clinical event committee.

11. A computational processing system for evaluating a clinical event, the system comprising:
a processor system; and
a memory system comprising one or more applications that can direct the processor system to:
receive at least one report associated with a patient within a clinical trial that has undergone a clinical event;
generate features from the at least one received report; and
enter the features into a predictive computational model to yield an evaluation of the clinical event.

12. The computational processing system of claim 11, wherein the clinical event is one of: an adverse event, a serious adverse event, an adverse reaction, or a suspected unexpected serious adverse event.

13. The computational processing system of claim 11, wherein the clinical event comprises: hospitalization or rehospitalization, disability, congenital anomaly, required intervention, allergic reaction, blood dyscrasias, seizures or convulsions, or development of drug dependence or drug abuse, death, or a cardiovascular related event.

14. The computational processing system of claim 13, wherein the cardiovascular related event is one of: transient ischemic attack (TIA), bleeding, myocardial infarction, arrythmia or conduction disturbances, structural valve deterioration, endocarditis, prosthetic valve dysfunction, thrombosis, and coronary obstruction.

15. The computational processing system of claim 11, wherein the clinical trial is assessing: a clinical procedure performed, a medical device utilized during a treatment, a prosthetic device, a system for performing clinical procedure, or a medicinal product.

16. The computational processing system of claim 11, wherein the at least one received report is a case report form, and wherein the features generated from the case report form comprises a categorical feature derived from a data entry.

17. The computational processing system of claim 11, wherein the at least one received report is a narrative or a source document, and wherein the features generated from the narrative or the source document comprises a categorical feature or a numerical value feature yielded from natural language processing.

18. The computational processing system of claim 11, wherein the predictive computational model is a regression-based model or a classification-based model.

19. The computational processing system of claim 11, wherein the evaluation of the clinical event is yielded in lieu of an adjudication by a clinical event committee.

20. The computational processing system of claim 11, wherein the evaluation of the clinical event is yielded to assess an adjudication by a clinical event committee.
